(19) United States
(12) Patent Application Publication
(10) Pub. No.: US 2024/0382317 A1
(43) Pub. Date: Nov. 21, 2024

(54) MODULAR LATERAL PLATING SYSTEM AND RELATED METHODS

(71) Applicant: GLOBUS MEDICAL, INC., AUDUBON, PA (US)

(72) Inventors: Brendan Horne, Avondale, PA (US); Bess Lorman, Harleysville, PA (US)

(21) Appl. No.: 18/317,956

(22) Filed: May 16, 2023

(51) Int. Cl. A61F 2/44; A61F 2/46

(52) U.S. Cl. CPC A61F 2/442; A61F 2/4603; A61F 2002/4615

(57) ABSTRACT

A bone plate having one or more screw holes configured to receive a coupling screw to attach the bone plate to a spacer, anchor holes configured to receive anchors or bone screws to attach the bone plate to one or more vertebral bodies, one or more blocking screws configured to allow passage of the bone screws into the plate body and to lock the bone screws in the plate body after passage, and a port to rigidly attach the bone plate to an inserter instrument. The bone plate allows rigid connection to a spacer that is implanted between adjacent vertebral bodies. The plate body may include one or more anchor holes.

### FIELD

The present disclosure relates to stable fixation of spine segments, allowing for fusion in, e.g., skeletally mature patients. More particularly, the disclosure relates to a bone fixation plating device that can be affixed to vertebrae of a spine and to a spacer inserted between adjacent spine segments. The invention also relates to a method for delivering and implanting the bone fixation plating device.

### BACKGROUND

Bones and bony structures are susceptible to a variety of weaknesses that can affect their ability to provide support and structure. Weaknesses in bony structures can have many causes, including degenerative diseases (e.g., degenerative disc diseases), tumors, fractures, dislocations and failed previous fusions. Some of these weaknesses can cause further conditions such as spondylolisthesis wherein bony structures slip out of their proper position.

In some cases of spinal surgery, it is known to use bone fixation plating devices (e.g., bone plate systems and rod and screw systems) to improve the mechanical stability of the spinal column and to promote the proper healing of injured, damaged or diseased spinal structures. Typically, corrective surgery can entail the removal of damaged or diseased tissue, a decompression of one or more neural elements, followed by the insertion of an interbody implant or bone graft for the purposes of a fusion or disc arthroplasty. In cases where spinal fusion is the desired surgical outcome, the surgery can often include implanting a bone plate or rod and screw system in order to immobilize adjacent vertebral bones to expedite osteogenesis across the vertebral segments.

Lateral lumbar interbody fusion (LLIF) and oblique lumbar interbody fusion (OLIF) are two types of minimally invasive spine fusion surgery in which surgeons access the spinal column through a lateral retroperitoneal approach. Diseased spinal discs are removed and supplemented with interbody devices to restore lost disc height and angle, as well as to provide stability so the segment can fuse and reduce nerve pain and discomfort. Stability of a spinal segment is critical for a successful fusion, and various types of implants can be used to facilitate fusion.

Plates have been used frequently for stabilization in the lumbar spine. Plating is often used in conjunction with spinal fusion to add stabilization to the segment. However, vertebrae and different surfaces on those vertebrae have varying shapes which causes variation in the desirable plate contour and screw trajectory. Improper placement and fit of a plate onto the vertebral bodies can weaken fixation and can also cause damage to the surrounding soft tissue and vasculature.

Additional issues include intra-operative movement in a plate during intra-operative placement and screw prep and to assist in the reduction of migration for the implant's life. There also exists a need for means of data collection on the forces in the segment allow for aid in placement intra-operatively and for patient monitoring post-operatively.

Accordingly, there is a need for a lateral plate that can be secured to vertebral bodies to reduce segment motion and promote stability. There is a need for a plate that can also be attached directly to the interbody to further reduce motion to decrease the likelihood of implant migration while further optimizing fusion potential.

### SUMMARY

In accordance with the present disclosure, a spinal fixation system for fusion of adjacent vertebral bodies including a bone plate having a screw hole, one or more anchor holes, a blocking screw for each of the anchor holes, and a port. The spinal fixation system further includes a spacer to attach to the bone plate via a coupling screw threaded into the screw hole and a bone fixation device to be inserted into each of the anchor holes to fixate the bone plate to one or more of the adjacent vertebral bodies.

In accordance with the present disclosure, a method of treating a spine for fusing adjacent vertebral bodies, the method including preparing a disc space between the adjacent vertebral bodies and providing a spinal fixation system. The spinal fixation system including a bone plate having a screw hole, one or more anchor holes, a blocking screw for each of the anchor holes, and a port. The spinal fixation system further includes a spacer to attach to the bone plate via a coupling screw threaded into the screw hole and a bone fixation device to be inserted into each of the anchor holes to fixate the bone plate to one or more of the adjacent vertebral bodies. The method further including inserting the spacer into the disc space, attaching the bone plate to the spacer, and fixating the bone plate to one or more of the adjacent vertebral bodies.

### BRIEF DESCRIPTION OF THE DRAWINGS

The following detailed description and examples are provided for the purpose of non-exhaustively describing some, but not necessarily all, examples or embodiments of the disclosure, and shall not limit the scope of the disclosure in any way.

FIG. 1 shows a top view of a bone plate consistent with the principles of the present disclosure.

FIG. 2 shows a top view of a bone plate consistent with the principles of the present disclosure.

FIG. 3 shows a top view of a bone plate consistent with the principles of the present disclosure.

FIG. 4 shows a top view of a bone plate consistent with the principles of the present disclosure.

FIG. 5A shows a shows a coupling screw to attach a bone plate to a spacer consistent with the principles of the present disclosure.

FIG. 5B shows a coupling screw to attach a bone plate to a spacer consistent with the principles of the present disclosure.

FIG. 6A shows a bone anchor or shim to attach a bone plate to a vertebral body consistent with the principles of the present disclosure.

FIGS. 6B-6F show bone screws to attach a bone plate to a vertebral body consistent with the principles of the present disclosure.

FIG. 7 shows an underside of the bone plate of FIG. 2 consistent with the principles of the present disclosure.

FIG. 8 shows an underside of the bone plate of FIG. 4 consistent with the principles of the present disclosure.

FIGS. 9A and 9B show perspective views of a bone plate attached to a spacer consistent with the principles of the present disclosure.

FIGS. 10A and 10B show perspective views of a bone plate attached to a spacer consistent with the principles of the present disclosure.

FIG. 11 shows a top view of a bone plate consistent with the principles of the present disclosure.

FIG. 12 shows an inserter consistent with the principles of the present disclosure.

### DETAILED DESCRIPTION OF THE EMBODIMENTS

It should be understood that this disclosure is not limited to the particular apparatus, methodology, protocols, and systems, etc., described herein and as such may vary. The terminology used herein is for the purpose of describing particular embodiments only and is not intended to limit the scope of the present disclosure, which is defined solely by the claims.

Described in further detail in the figures, lateral plates have a low-profile design in one-hole, two-hole, and four-hole configurations that will integrate directly with non-integrated LLIF and OLIF implants. The low-profile design of the lateral plates allows for minimal opening of the retractor, which will help reduce retraction on critical soft tissue anatomy. The lateral plates can directly connect to the interbody will help increase the overall stability of the segment to further optimize fusion potential. The new lateral plates will accommodate all variations of LLIF including OLIF and prone lateral procedures, and can incorporate navigation to allow surgeons to visualize plate placement without fluoroscopy.

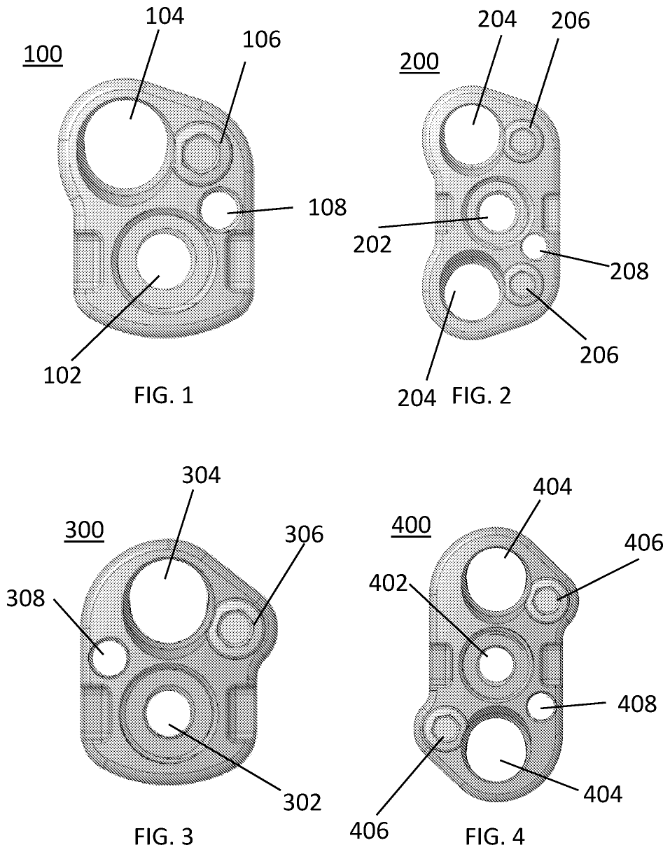

Turning to FIGS. 1-4, illustrated are two sets of lateral plates, one that integrated with static LLIF/OLIF implants and another that integrated with expandable LLIF/OLIF implants. FIGS. 1 and 3 illustrate lateral plates that have a one-hole design. Plate 100 of FIG. 1 has a screw hole 102, an anchor hole 104, blocking screw 106, and a port 108. Plate 300 of FIG. 3 has a screw hole 302, an anchor hole 304, blocking screw 306, and a port 308. FIGS. 2 and 4 illustrate lateral plates that have a two-hole design. Plate 200 of FIG. 2 has a screw hole 102, anchor holes 204, blocking screws 206, and a port 208. Plate 400 of FIG. 4 has a screw hole 402, anchor holes 404, blocking screws 406, and a port 408. With these plates, surgeons will have the option to use the lateral plate regardless of their implant preferences. The one- and two-hole lateral plates are designed to connect to non-integrated static LLIF and OLIF interbody devices.

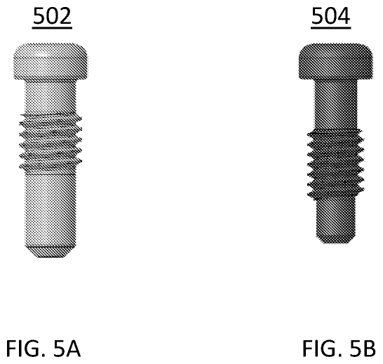

FIG. 1 will be described in further detail and it is noted that the description of the screw hole, anchor hole, blocking screw, and port of FIG. 1 also applies to these components of the other plates 200, 300, and 400. FIG. 1 illustrates a plate 100 having screw hole 102, anchor hole 104, blocking screw 106, and port 108. Screw hole 102 is configured to receive a coupling screw that couples plate 100 to an interbody device as described in more detail below. For example, illustrated in FIGS. 5A and 5B are coupling screws 502 and 504 that may be inserted into screw hole 102 to couple plate 100 to an interbody device. This will reduce the potential for interbody migration and increase segment stability.

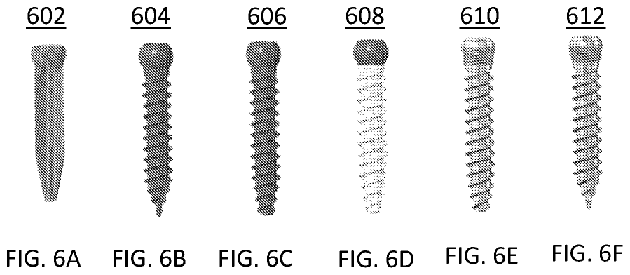

Anchor hole 104 is configured to receive a bone screw or a bone anchor that secures plate 100 to one or more vertebral bodies. Illustrated in FIGS. 6A-6F are exemplary bone fixation devices. FIG. 6A illustrates a bone anchor or a shim 602 and FIGS. 6B-6F illustrate bones screws 604, 606, 608, 610, and 612. Each of shim 602 and screws 604-612 may be inserted into anchor hole 104 to secure plate 100 to a vertebral body. In this manner, screw hole 104 on new lateral plate 100 will accept a variety of fixation options including self-drilling bone screws, self-tapping bone screws, variable and fixed angle bone screws, bone anchors and HA coated screws.

Blocking screw 106 is an external blocking mechanism that will help secure bone screws/anchors to the vertebral bodies, in an attempt to prevent backout. The blocking screws are configured to allow passage of the bone screws into the bone plate in a first position and to lock the bone screws in the plate body after passage in a second position by, for example, rotating the blocking screw. Port 108 may be a universal threaded port that will allow for an external threaded instrument to be attached to assist with plate insertion, in situ handling, or removal.

As noted above, FIGS. 1 and 3 illustrate plates that accept one bone fixation device to engage a vertebral body. FIGS. 2 and 4 contain similar components and have a two-hole design to accept a bone fixation device in each anchor hole to, for example, engage adjacent vertebral bodies across a joint or intervertebral space while an implant is secured in the intervertebral space.

The design of the lateral plates 100, 200, 300, and 400 (including one- and two-hole plates for both static and expandable interbody devices) ensure optimal usability, and will allow surgeons to insert the plate either already attached to the interbody, or in situ after the interbody has been placed. This flexibility allows surgeons to implement whatever decision is best for their workflow and patient.

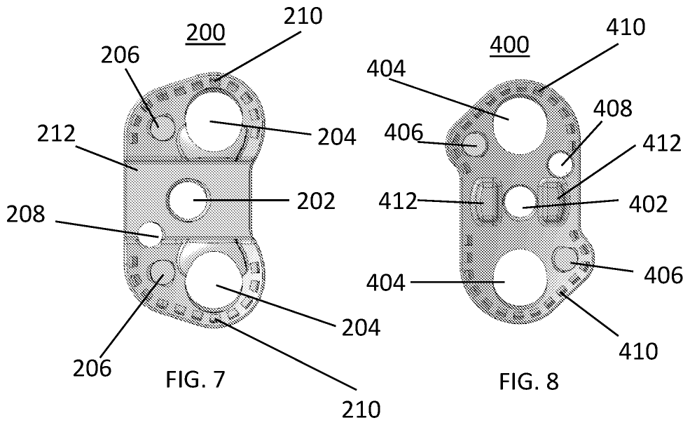

Turning now to FIGS. 7 and 8, illustrated are the backsides of two-hole plates 200 and 400, respectively. The backside of the plates feature a toothy ridge to act as an initial point of stabilization for the plate on the bone. This is illustrated as ridges 210 on plate 200 and ridges 410 on plate 400. The one-hole plates 100 and 300 may also have similar ridges for this purpose. The teeth may bite into the vertebral bodies to keep the plate from shifting during screw prep if the coupling screw is not used and the plate remains un-attached from the compatible interbody spacer. The teeth also when gripped into the vertebral body act as point of fixation resisting axial torsion for the two-hole plate.

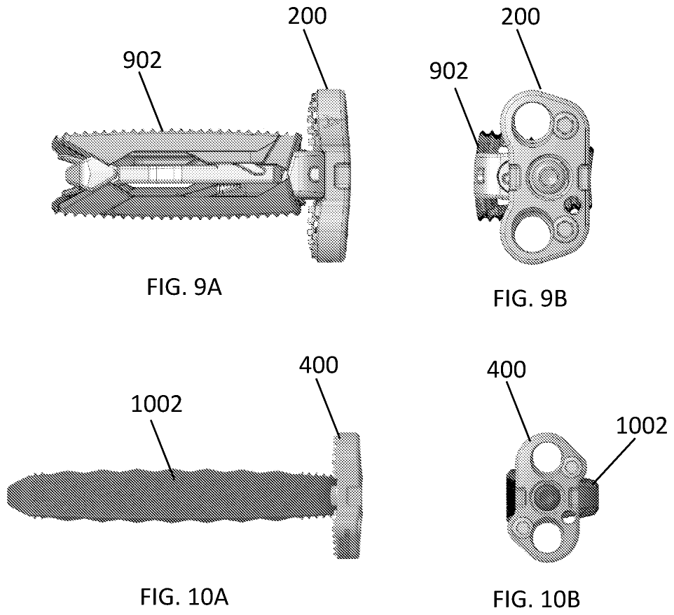

The lateral plates may have features for engaging intervertebral bodies when the two are attached. For example, plate 200 may have a flat cutout 212 on the back of plate 200 that may attach to a spacer, such as expandable spacer. The inferior and superior sides of cutout 212 may engage with a back ramp of the expandable spacer, which aids in plate alignment to the spacer in-situ. For example, FIGS. 9A and 9B illustrate perspective views of plate 200 engaged with a spacer 902. Plate 200 may be directional and marked with an A on the anterior side of the plate. One-hole plate 100 may be designed to be used only at the superior vertebral body.

Plate 400, for example, may also have features for engaging a spacer that include two prongs 412 that sit in an inserter pocket of plate 400. Plate 400 may be configured so that it is not directional and may be used with either bone screw hole at the superior or inferior vertebral body and the compatible one-hole plate (plate 300) may be used at either vertebral body.

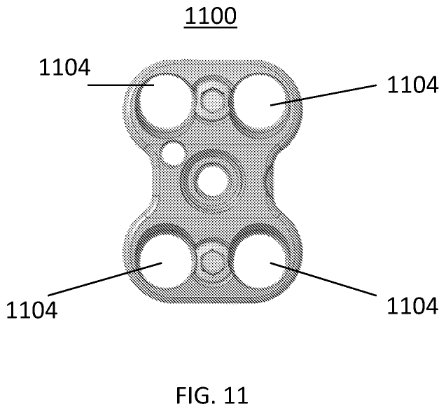

Turning to FIG. 11, illustrated is a 4-hole lateral plate 1100 that can be used in conjunction with a spacer, for example, expandable spacer 902. Plate 1100 may be similar to plates 300 and 400 with similar components. Plate 1100 may also have a cutout to engage with spacer 1002, including having ridges 410 to engage bone. Instead of either one or two bone fixation devices to engage bone, plate 1100 has four anchor holes 1104 to receive bone fixation devices as previously described. In addition, a 4-hole anchor plate may have features described with plates 100 and 200 to engage an expandable spacer such as spacer 902.

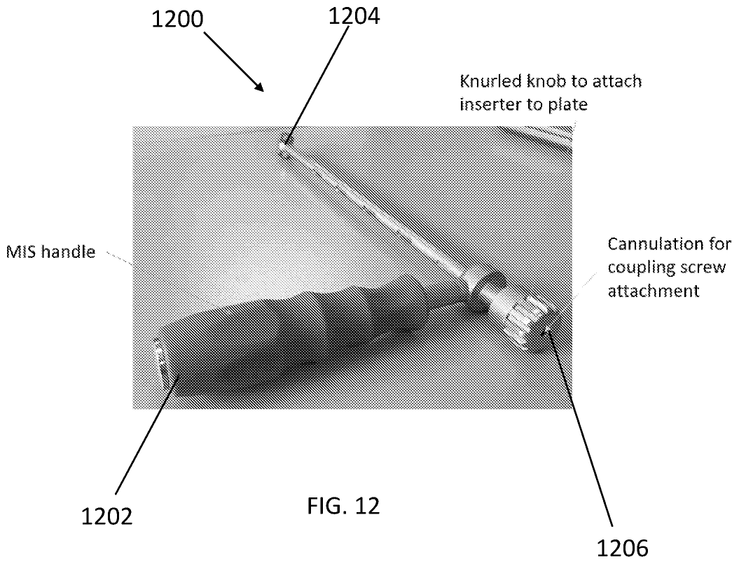

FIG. 12 illustrates a rigid inserter 1200 for each of the plates described above and threads into the ports of each of the plates. Inserter 1200 has a modular MIS handle 1202 for improved surgeon handling, and a threaded attachment 1204 with the plate to improve the strength of the connection between parts. The inserter allows for a threaded connection 1204 between the plate and the instrument, but also has a cannulated shaft 1206 which enables the surgeon to introduce a secondary driver to turn the coupling screw. Having a single instrument to hold and attach the plate to the interbody simplifies surgeon workflow by reducing the number of required instruments and number of procedural steps.

It is intended that all matter contained in the above description or shown in the accompanying drawings shall be interpreted as illustrative only and not limiting. Changes in detail or structure may be made without departing from the spirit of the disclosure as defined in the appended claims.

What is claimed is:

1. A spinal fixation system for fusion of adjacent vertebral bodies comprising:
a bone plate having a screw hole, one or more anchor holes, a blocking screw for each of the anchor holes, and a port;
a spacer configured to attach to the bone plate via a coupling screw threaded into the screw hole; and
a bone fixation device configured to be inserted into each of the anchor holes to fixate the bone plate to one or more of the adjacent vertebral bodies.

2. The spinal fixation system of claim 1, wherein an underside of the bone plate contains a toothy ridge configured to engage one of the adjacent vertebral bodies.

3. The spinal fixation system of claim 1, wherein an underside of the bone plate contains a cutout configured to receive a portion of the spacer.

4. The spinal fixation system of claim 1, wherein an underside of the bone plate contains one or more prongs that are received in a portion of the spacer.

5. The spinal fixation system of claim 1, wherein the port is threaded.

6. The spinal fixation system of claim 6, wherein the port is configured to receive a threaded end of an inserter instrument.

7. The spinal fixation system of claim 1, wherein each blocking screw is configured to rotate from a first position to a second position to lock one bone fixation device to the bone plate.

8. The spinal fixation system of claim 1, wherein each bone fixation device is a bone screw.

9. The spinal fixation system of claim 1, wherein each bone fixation device is a shim.

10. The spinal fixation system of claim 1, further comprising an inserter configured to attach to the bone plate via the port.

11. A method of treating a spine for fusing adjacent vertebral bodies, said method comprising:
preparing a disc space between the adjacent vertebral bodies;
providing a spinal fixation system including:
a bone plate having a screw hole, one or more anchor holes, a blocking screw for each of the anchor holes, and a port;
a spacer configured to attach to the bone plate via a coupling screw threaded into the screw hole; and
a bone fixation device configured to be inserted into each of the anchor holes to fixate the bone plate to one or more of the adjacent vertebral bodies,
inserting the spacer into the disc space;
attaching the bone plate to the spacer; and
fixating the bone plate to one or more of the adjacent vertebral bodies.

12. The method of claim 11, wherein an underside of the bone plate contains a toothy ridge configured to engage one of the adjacent vertebral bodies.

13. The method of claim 11, wherein an underside of the bone plate contains a cutout configured to receive a portion of the spacer.

14. The method of claim 11, wherein an underside of the bone plate contains one or more prongs that are received in a portion of the spacer.

15. The method of claim 11, wherein the port is threaded.

16. The method of claim 16, wherein the port is configured to receive a threaded end of an inserter instrument.

17. The method of claim 11, wherein each blocking screw is configured to rotate from a first position to a second position to lock one bone fixation device to the bone plate.

18. The method of claim 11, wherein each bone fixation device is a bone screw.

19. The method of claim 11, wherein each bone fixation device is a shim.

20. The method of claim 11, further comprising an inserter configured to attach to the bone plate via the port.
